(19) United States
(12) Patent Application Publication
(10) Pub. No.: US 2019/0147992 A1
(43) Pub. Date: May 16, 2019

(54) Electronic Healthcare Treatment Discharge System

(71) Applicant: National Health Coalition, Inc., Fort Worth, TX (US)

(72) Inventors: Kenneth A. Hill, SR., Fort Worth, TX (US); Clinton S. Ferguson, III, Arlington, TX (US)

(73) Assignee: National Health Coalition, Inc., Fort Worth, TX (US)

(21) Appl. No.: 16/176,963

(22) Filed: Oct. 31, 2018

Related U.S. Application Data
(60) Provisional application No. 62/580,217, filed on Nov. 1, 2017.

(51) Int. Cl. G16H 15/00; G16H 10/60

(52) U.S. Cl. CPC G16H 15/00; G16H 10/60

(57) ABSTRACT

A system for preparing in electronic form a discharge summary (EDS) having at least information regarding patient identity, new prescriptions, medications to be discontinued, and clinical follow-up. The EDS is to be delivered to the identified patient upon discharge from a treatment facility. The system and process uses first and second data processors operating according to programmed instructions that are linked to corresponding databases and a network of pharmacies to ensure all information in the EDS is delivered to the patient and the medications to be discontinued, including prescription and non-prescription medications, are properly documented in the pharmaceutical records of the patient.

### CROSS REFERENCE TO RELATED APPLICATIONS

The present application is also related to U.S. Provisional Patent Application Ser. No. 62/580,217 filed by the same inventor and entitled ELECTRONIC HEALTHCARE TREATMENT SUMMARY DEVICE, incorporated in its entirety herewith. The present application is also related to U.S. Provisional Application Ser. No. 62/393,365 filed by the same inventor Sep. 12, 2016 and entitled HEALTHCARE DATA SYSTEM AND PROCESSES, incorporated in its entirety herewith. The present application is also related to the following U.S. Patent Applications filed Sep. 8, 2017 under Prioritized Examination and incorporated herein in their entirety: Ser. No. 15/699,772 entitled SYSTEM FOR PROCESSING IN REAL TIME HEALTHCARE DATA ASSOCIATED WITH SUBMISSION AND FULFILLMENT OF PRESCRIPTION DRUGS; Ser. No. 15/699,827 entitled PROCESSING PHARMACEUTICAL PRESCRIPTIONS IN REAL TIME USING A CLINICAL ANALYTICAL MESSAGE DATA FILE; and Ser. No. 15/699,852 entitled METHODS FOR PROCESSING SUBMISSION AND FULFILLMENT OF PHARMACEUTICAL PRESCRIPTIONS IN REAL TIME.

### BACKGROUND OF THE INVENTION

1. Field of the Invention

The present invention generally relates to management and integration of database information from multiple sources and more particularly to an electronic, patient-centric, portable, interoperable, interdisciplinary healthcare database system. The system includes healthcare data associated with prescription drugs, clinical services, physician and hospital and other healthcare provider services, and is designed for improving and reporting patient outcomes and providing processes and mechanisms for secure access by patients and their authorized healthcare providers.

2. Background of the Invention and Description of the Prior Art

It is widely known that the healthcare industry is highly complex. Massive amounts of data are involved in documenting every episode of care, and related transactions. And now the outcome reporting associated with every patient required by the Center for Medicare and Medicaid Services (“CMS”) adds to that complexity. Numerous healthcare providers participate in each episode of care for a patient. Healthcare service providers, including pharmacies, physicians, hospitals, laboratories, clinics, medical institutions, and regulatory agencies that develop clinical standards of care historically have operated their own data systems, typically according to unique and different formats and processes.

An essential participant in healthcare services is another entity, the “payer” often an insurer, or employers, which pays for the services rendered and the claims made by the other healthcare providers. The insurance system includes the insurance companies that provide funds for payments to providers and to intermediaries that submit, adjudicate and facilitate payment transactions for services rendered to patients. In many instances it is difficult to timely access and use important data for patients' care and their outcomes. With regulatory requirements that affect payments to healthcare providers and require reporting of patient measurable outcomes, further complexity and difficulties are assured if not adequately addressed.

The population of the United States, at the end of June, 2016, exceeded 324 million persons. Every person is a patient or potentially a patient of the healthcare system that requires medical attention and treatment, each with his or her unique healthcare needs and genetic profile, susceptibility to disease, and medical history. Many people contract an illness or are injured at some time during their lifetimes. The volume of patient healthcare data that accumulates during a patient's lifetime is enormous. Likewise, the data records of all of the healthcare providers who treat the patients, the data records of business entities including insurance companies and employers involved in payment transactions on behalf of the patients, and the records of governmental agencies involved in benefit programs and regulatory requirements associated with the healthcare of the patients adds to the volume of data.

The need for timely access and processing of this massive data is essential to ensure that adequate healthcare services are delivered when needed and that the transactions and data associated with those services to patients are updated and completed with minimal delay. There is also then a need to promptly process reported measureable outcomes for treatment services, and ultimately payment.

An efficient healthcare system that maintains the health of persons active in the nation's economic enterprise is vital to its economic system. To accomplish the patient care required, it is essential that the data processing needs of all participating elements of the healthcare system be available to fulfill the demands of the healthcare system for timely access and handling of the massive amounts of data generated by the healthcare system. This is true for effective, cost-efficient healthcare services, and appropriate timely payment for same.

Handwritten record-keeping is clearly insufficient for these complex tasks. And the data processing systems operated by individual participants in the healthcare system have previously struggled to handle the tasks of keeping timely records, even with the aid of intermediaries that facilitate data interchange among participants. The ability to process the complex transactions involved in healthcare, and particularly for maintenance of patient records and for reimbursement of the fees and costs billed by providers—physicians, hospitals, laboratories and pharmacists—has accelerated faster than the capability to document and measure outcomes of the healthcare services (“Outcomes”) to the patients. Assurance of adherence to standards of care and improved efficiencies are now required by newly applicable regulations and must be realized with minimal delay.

Actions of the Federal Government in recent years have supplied some of the impetus toward upgrading the efficiency of the healthcare delivery system in the United States. Such actions include the Omnibus Budget Reconciliation Act of 1990 (OBRA '90), which requires drug utilization review procedures; an executive order by President George W. Bush in April, 2004, which set a ten year goal for “deployment of health information technology within 10 years;” and establishing the Health Information Technology for Economic and Clinical Health Act (HITECH Act), which sets forth “meaningful use of interoperable Electronic Health Records.” The HITECH Act, enacted as part of the American Recovery and Reinvestment Act of 2009, includes numerous categories of patient health records. And recently, the Center for Medicare and Medicaid Services (CMS) has established requirements for reporting patient outcomes of healthcare services delivered to patients in connection with approval for payments to providers.

What is needed is a comprehensive integration of the systems for obtaining and processing and utilizing healthcare patient data. To accomplish this task with minimal delay and with ways that are adaptable to the growth of the population, and to the needs of patients, providers and business organizations that serve the healthcare system, it is only possible by the use of highly developed computer systems operable according to program instructions that are configured in scalable ways for these essential tasks. There is no technology other than computers, networks, databases, and systems for processing such a volume of data (“big data”) that will accommodate the need for data processing in the healthcare system. The need for continued innovation and improvement of the technology of the comparing elements themselves, including particularly the software or program instructions, is essential for the healthcare system to succeed in delivering the needed healthcare services and to meet the governmental regulatory requirements.

Innovation in healthcare delivery is a never-ending requirement. Well-developed software must be utilized to run efficiently to this end. Software and systems must be faster, with more efficient architectures and innovative ways to organize and structure the data processing environment.

### SUMMARY OF THE INVENTION

A system is provided for preparing an electronic discharge summary (EDS) to an identified patient upon discharge from a healthcare treatment facility, comprising a first pharmacy for receiving an EDS that includes a new electronic prescription for a pharmaceutical issued by a healthcare provider in the healthcare facility via an electronic medical record (EMR) intermediary to a first data processor, wherein the EDS, after a review by the first data processor, is forwarded to the first pharmacy to compare medications to be deactivated by the EDS with prescription records of the identified patient with a prescription history record in other pharmacies associated with the first pharmacy and having prescription records of the identified patient; a first data processor configured by first programmed instructions to receive the EDS issued by the healthcare provider via the EMR intermediary and process instructions in the EDS to discontinue the medications to be deactivated by comparisons with first information regarding previous prescriptions dispensed to the identified patient received from a network of other pharmacies including the first pharmacy that have access to prescription records of the identified patient and with second information from a consolidated database in an electronic patient outcome record (EPOR) data system; and a second data processor coupled to the consolidated database and to the first data processor, and configured by second programmed instructions to receive the first and second information about the identified patient's prescription history from the network of pharmacies and from the consolidated database, to confirm that medications to be deactivated are discontinued, and to provide the EDS to the EMR for transmission to the first pharmacy for dispensing the new prescription and the EDS to the identified patient.

In other aspects the system includes a pharmacy identified by the identified patient, i.e., the patient's pharmacy; that the EDS includes at least one new prescription, a list of medications to be discontinued, and clinical follow-up instructions; that the medications to be deactivated includes previous prescription and non-prescription medications; and that the healthcare provider includes a physician, a nurse practitioner, or other authorized healthcare professional in the healthcare facility.

In another embodiment a process is disclosed for providing an electronic discharge summary (EDS) to an identified patient upon discharge from a healthcare treatment facility, comprising the steps of receiving the EDS and at least one new electronic prescription included therein for a diagnosed condition of the identified patient as issued by a healthcare provider in the healthcare facility in the name of the identified patient and transmitted via an electronic medical record (EMR) intermediary to a first data processor configured by first programmed instructions; reviewing in the first data processor according to the first programmed instructions the EDS to identify the at least one new electronic prescription and medications listed in the EDS to be deactivated and to confirm the presence of clinical follow-up instructions associated with the diagnosed condition stated in the EDS; parsing the contents of the EDS in the EPOR into its components and forwarding each component to a first pharmacy for comparing the medications to be deactivated with first information regarding previous prescriptions dispensed to the identified patient received from a network of other pharmacies including the first pharmacy that have access to prescription records of the identified patient, and with second information from a consolidated database in an electronic patient outcome record (EPOR) data system; sending notices to the network of other pharmacies that the previous prescriptions and medications included in the list of medications to be deactivated are to be discontinued; notifying healthcare providers to schedule appointments for providing the clinical follow-up according to the clinical follow-up instructions in the EDS; preparing the new prescription and the accompanying EDS for dispensing to the patient; and comparing the EDS with the first and second information in a second data processor configured according to second programmed instructions to confirm that medications to be deactivated are discontinued, and to provide the EDS to the EMR for transmission to the first pharmacy for dispensing the new electronic prescription and the EDS to the identified patient.

In another aspect, the process includes confirming presence of required components of the EDS including patient identity, condition diagnosed, new prescription and non-proscription medications, medications to be deactivated and discontinued, and clinical follow-up instructions.

In other aspects, the process includes filling the new prescription for dispensing it to the identified patient; and delivering the EDS and the new prescription and associated consultation as needed about the new prescription, the deactivated medications, and the clinical follow-up instructions contained in the EDS.

In other aspects, the process includes confirming to the EPOR data system the comparison of the EDS with the first and second information; and updating the EPOR data system with a message stating the new prescription has been dispensed, the discontinued medications deactivated, and the clinical follow-up instructions delivered and scheduled.

### BRIEF DESCRIPTION OF THE DRAWINGS

FIG. 1 illustrates a system functional components network diagram depleting major of one embodiment of the present invention;

FIG. 2 illustrates a functional block diagram of front end processing of a specialty prescription drug according to one embodiment of the invention;

FIG. 3 illustrates a functional block diagram of back end processing of a specialty prescription drug according to the embodiment of FIG. 2;

FIG. 4 illustrates an alternate embodiment of the invention depicted in FIGS. 1, 2 and 3 for providing reports generated from the consolidated EPOR database;

FIG. 5 illustrates a block diagram of a data processor according to an embodiment of the present invention;

FIG. 6 illustrates a system block diagram of another embodiment of the present invention; and

FIG. 7 illustrates a flow chart of one process that can be carried out in the embodiment of FIGS. 5 and 6.

### DETAILED DESCRIPTION OF THE INVENTION

The advancement in technology described herein meets the need for real time processing, updating, and accessing of the massive amounts of data involved in the delivery of healthcare. It is a system best characterized as a new, real time “Patient-Centric, Portable, Interoperable, Interdisciplinary, Electronic Patient Outcome Record” that is produced by and functions cooperatively within a new arrangement of principal components in a healthcare data system that resides in an electronic healthcare network. It is a network that links participants in the system—patients and authorized healthcare providers, data resources and processing elements, payers, pharmaceutical manufacturers, and pharmacies—in the delivery of healthcare services to patients through designated portals with each other and with the comprehensive database system. One of several chief features of the system is that it ensures safe, secure, real time access to a comprehensive Electronic Patient Outcome Record (“EPOR”) by authorized healthcare providers including their patients through portals to the databases within the network. The data resources including the EPOR are updated in real time with every healthcare transaction to ensure the availability of timely, secure, and accurate information to the participating healthcare providers and their patients. This feature alone ensures patient safety.

The system is structured around a network of three database components and a clinical services platform that includes specially developed software including program instructions and analytics directed to rapid pre-edit reviews of prescriptions against all available pharmaceutical and clinical information, consolidating the results of all healthcare data associated with each pharmaceutical transaction, and enabling the access to the consolidated data in real time to all authorized healthcare providers. The three database components include a structured healthcare database of standardized healthcare data and pharmaceutical data called electronic patient outcome data (“EPO data”) [1^{st} component]; an electronic pharmacy record of the drug prescription data contained in the collective databases of pharmacies (“ePR”) database—an intra-pharmacy record—[2^{nd} component]; and an electronic patient outcome record (“EPOR”) database [3^{rd} component] that is a comprehensive database populated by data from the EPO data and ePR databases and electronic prescription transactions. The specially developed software that provides operational control is embodied in the new clinical services platform. Additional software is embodied in a pharmaceutical delivery system that interfaces with a national Health Information network (“NHIN”) to supply data and services to the EPO database and process claims for payment for services.

The system described herein integrates the data storage and processing facilities of the healthcare industry in a way that achieves its real time operability through the use of specialized software systems—generally residing in virtual computing systems such as cloud storage to be described—and accessible throughout the system from strategic locations. The computer systems that provide access to this network are necessarily distributed throughout the healthcare system. This real time interoperability is organized, in part, around the processes and transactions involving the prescribing of ethical drugs by physicians for treatment, delivery of the drugs through the pharmaceutical system, and processing the payments to healthcare providers and pharmacies for healthcare services rendered, all according to standardized rules and procedures established by industry practice and in compliance with federal regulatory requirements for documenting and reporting measurable patient outcomes.

The new structures of the system build on some existing elements of the healthcare system, primarily those that are configured for processing and documenting the delivery of healthcare services and products by providers. These providers include physicians, hospitals, laboratories and pharmacies, which provide healthcare services and products and seek reimbursement from payers for billed fees and expenses for those services and products. The new structure integrates the existing elements with the new electronic healthcare system components described herein. The system provides for an electronic patient outcome record (EPOR) database, bidirectional communication portals into the system and the ePR and EPO data databases, and the clinical services platform that may incorporate a review processor and access to program instructions for standardized access. The resulting combination provides the aforementioned “Patient-Centric, Portable, Interoperable, Interdisciplinary, Electronic Patient Outcome Record”. This system operates to manage the processing of prescription drugs, from a full and complete automatic submission review including pre-edits of the electronic prescription followed by automatic fulfillment review including delivery of essential clinical and educational services, dispensing of the pharmaceutical to the patient, and processing and editing of the claims for payment or reimbursement. The system also documents data associated with the delivery of healthcare services including patient outcomes as needed for payment as required by the Center for Medicare & Medicaid Services (“CMS”). In other embodiments, the system is adapted to provide an electronic discharge summary to a patient upon discharge from treatment, or an electronic admission summary—or a Medication Reconciliation Summary—to providers at the time of admission or during an episode of care with a provider for evaluation or treatment.

The features and benefits of the system could not be achieved in real time without the program instructions and analytical processes—software specifically designed to ensure that this processing provides continually updated data and access to it throughout every patient—provider encounter and transaction associated with each episode of healthcare provided to each patient. A further key component of the system to be described is an innovative data file implemented in a message structure that travels with and is involved in the transactions processed by the system. As is well known in the art, a data file, in one example, is a component of a data record comprised of a sequence of fields for the entry of data. A database is a storage repository for data records. In the present system the new “Clinical Analytical Message” or “CAM” is an integral part of the system that enables efficient communication of patient and prescription information in the accurate and complete, real time processing of the submission and fulfillment transactions, especially when involving specialty prescription drugs. Moreover, these features and benefits would not be possible without the combination of the high speed capabilities of computer processing, direct access to the various databases in the system through the designated portals and networks within the system, and the reliance on redundant data structures and extensive use of cloud storage. Thus, the system to be described relies on a combination of data processing mechanisms, both hardware and software, to configure the system to achieve the data processing results in real time—i.e., at nearly the same time as the system is accessed to enter a prescription submission or check on the status of a patient record, for example.

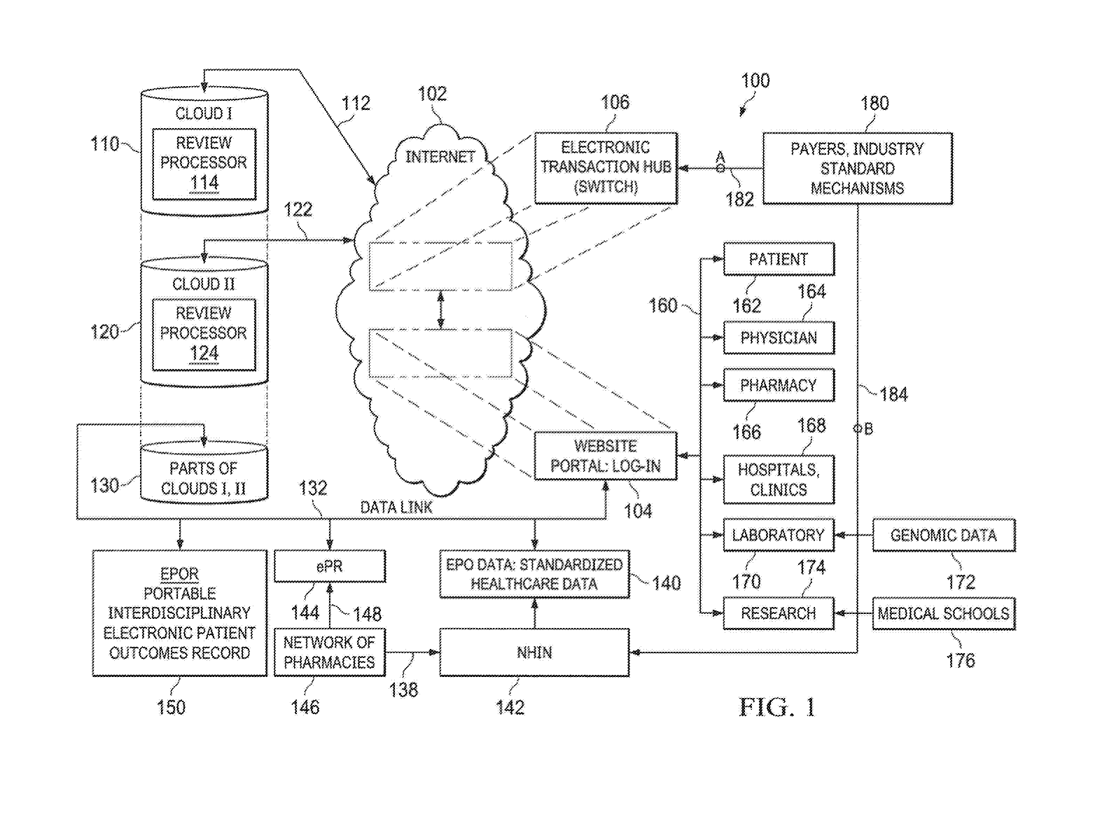

The present invention thus provides a new system architecture for processing healthcare data that is depicted in FIGS. 1, 2, and 3. The invention comprises a combination of data processing components arranged and configured for efficiently processing both prescription submissions and fulfillment, as well as processing healthcare data updates and secure patient outcome data accessibility in real time to all authorized healthcare providers and patients. The technological improvements embodied in the present invention include configuring the following three innovations: (1) an enhanced database architecture; (2) an enhanced (automated) prescription submission and fulfillment processing system provided by a new clinical services platform; and (3) a data file or message format for facilitating data movement and processing in both systems (1) and (2).

### Overview of Key System Concepts

It will be helpful to understanding the description that follows by briefly reviewing several key concepts embodied in the present invention. The concepts are: (1) real time access by all authorized participants in the healthcare system to complete clinical and pharmaceutical data for each patient, that is updated in a comprehensive electronic patient outcome record (EPOR) at each healthcare transaction; (2) complete pharmaceutical files for each patient includes, but is not limited to the descriptive information about the drug, interaction data with both other drugs and foods, and drug-specific laboratory and genomic information; and (3) an essential functional principle of the system is the capability to provide the clinical analytical message (“CAM”) by which key information is conveyed and delivered as needed during the process of prescribing, reviewing, distributing, and dispensing of specialty and non-specialty prescription drugs, for and to the patient, and managing and recording measurable outcomes related to treatment for the patient. An additional concept (4) of a patient specific algorithm that functions as a secure doorway of the patient's file along with the clinical analytical message is also discussed.

The real time accessibility of clinical and drug data means that the files of the EPO data database and the electronic pharmacy record (ePR) database are accessible and updated during read/write transactions by healthcare providers authorized to access the data. The EPOR, populated from the EPO data and ePR databases, is a comprehensive database consolidated from the EPO and the ePR data that is also be updated at each transaction to help ensure that accurate data is available for the next healthcare provider that accesses the record so that healthcare provider actions and decisions are informed by the data. This concept helps assure patient safety by maintaining timely updates to the patient record, a primary objective of the healthcare system disclosed herein. Regarding specialty drugs, the delivery of those drugs to a patient may not proceed or be completed unless a current, accurate, and comprehensive patient data record is available and satisfies the clinical requirements for such fulfillment. Real time updates to these data bases also facilitates ongoing healthcare research evaluation of required clinical data.

The completeness of pharmaceutical and clinical files, created for each patient, is essential to enable prescribing specialty drugs that are compatible with the metabolic and clinical profile of the individual patient to help ensure (a) that all laboratory data is accessible; (b) that relevant genomic data is available; (c) that a drug or dosage that can be adequately and safely metabolized will be prescribed; (d) that side effects are minimized, including those that could be potentially dangerous; (e) that potentially dangerous interactions with other drugs or foods are minimized; and (f) that the most effective drug is prescribed. The pharmaceutical drug files as configured in the system transcends the need for formularies—tiers and lists of different categories of branded and generic drugs used by payers to limit drug dispensing to the cheapest drug rather than the most appropriate drug.

In another application of the pharmaceutical and clinical data files, chain retailers offering both grocery and pharmacy products are excellent candidates for utilizing the benefits of these comprehensive pharmaceutical files that form an integral part of the present healthcare system disclosed herein. The availability of such files, when incorporated into a retail environment that also provides, for example, food products purchased by the patient, enables interaction cross checks to be performed easily from data resident in the same database system used by the retail business. This analysis capability helps to avoid dispensing drugs incompatible with the patient's diet and clinical profile, and with other substances that may be consumed by the patient.

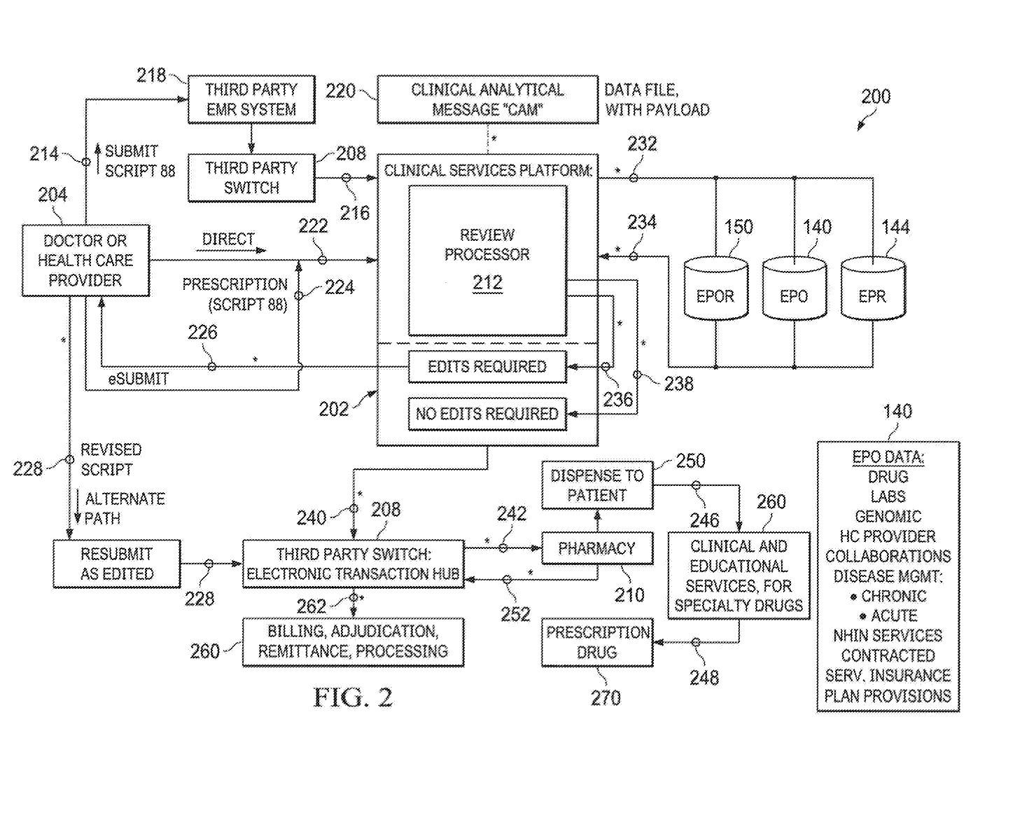

The use of a clinical analytical message (CAM)—which may be structured as a data file—also provides an essential adjustment and/or confirmation function during the process of fulfilling a prescription. The term clinical means that the message includes the relevant known patient clinical data to help ensure that the supplied patient data is consistent with other information involved in the transaction taking place during the fulfillment process. The term analytical means that the drug data in the patient's file—description, genomic, interactions, and even laboratory data, etc.—that is specific to the patient for whom the prescription is being processed is analyzed for consistency with the clinical data that relates to the prescription. The term message means that the CAM includes all relevant prescription and patient clinical data as well as the educational and clinical services information that must be delivered to the patient and to the electronic patient outcome record during the fulfillment process is readily available and communicated so that compliance with FDA and CMS requirements may be met during the steps of the fulfillment process. Details of the CAM data file are included in the description of FIG. 2 below.

One additional feature of the healthcare data system disclosed herein is the role of a patient specific algorithm (“PSA”) created for and unique to each patient. This algorithm functions as a unique identifier, an address—or secure doorway—of the patient's file as it is accessed, retrieved, and updated by authorized users—the patient and authorized healthcare providers such as physicians, hospitals, pharmacists, laboratories, and payers. The PSA, which may be associated with a message header incorporated in the CAM data file, includes data security features that help prevent unauthorized access to the patient's data file.

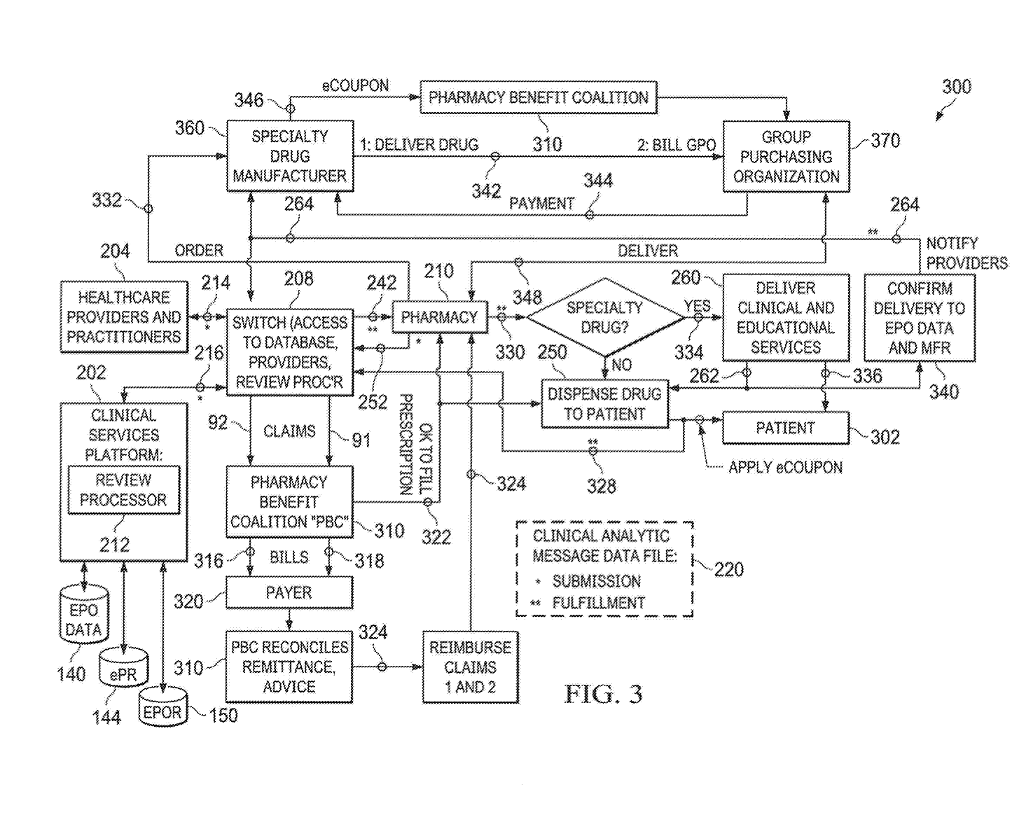

FIG. 1 illustrates abroad overview of the principal features of the system and network for processing prescriptions for pharmaceuticals. FIG. 2 illustrates the major process steps and functional components involved in a first embodiment—the submission review phase associated with reviewing a prescription up to and including submitting the prescription to a pharmacy for fulfillment. FIG. 3 illustrate the major process steps and functional components involved in a second embodiment—the fulfillment review phase associated with processing the prescription at the pharmacy including delivering required clinical and educational services information to the patient, dispensing the specialty drug to the patient, and processing payment transactions associated with delivering the clinical and educational services information and dispensing the prescription medication to the patient.

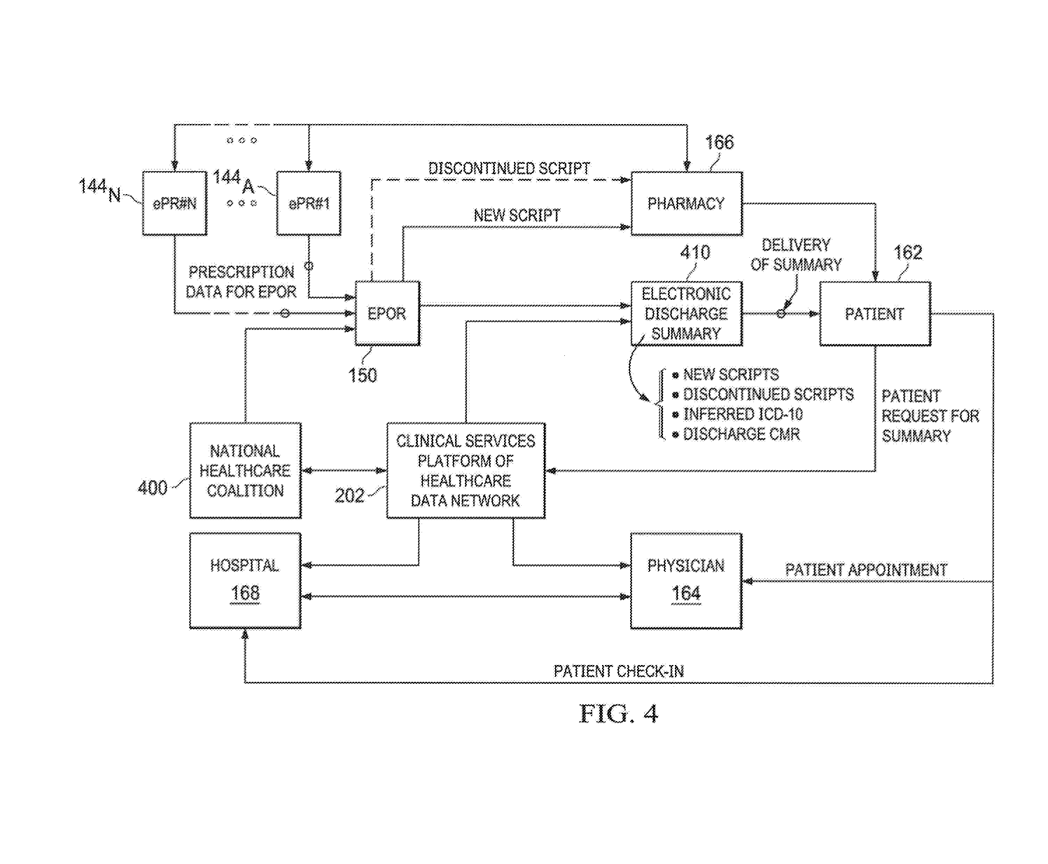

FIG. 4 illustrates one example of a third embodiment: the use of the system disclosed herein to provide an electronic hospital discharge summary. It is one of three examples of this use. Besides the electronic hospital discharge summary, an electronic admissions summary and a comprehensive medication reconciliation report may also be produced according to similar uses of the system disclosed herein. These uses exploit the real time availability of the consolidated patient record in the EPOR as configured in the system.

A key feature of these embodiments is a new comprehensive Electronic Patient Outcome Record (EPOR) 150 that consolidates data obtained from two other databases (the EPO Data 140 and the ePR data 144) during the processing of a prescription. The EPOR is maintained for access by any patient and practitioner, hospital and clinic, laboratory and medical research entity authorized to participate in the use of the system. The system is configured to update all database entries as each healthcare transaction takes place so that the data entry and access may be completed in real time.

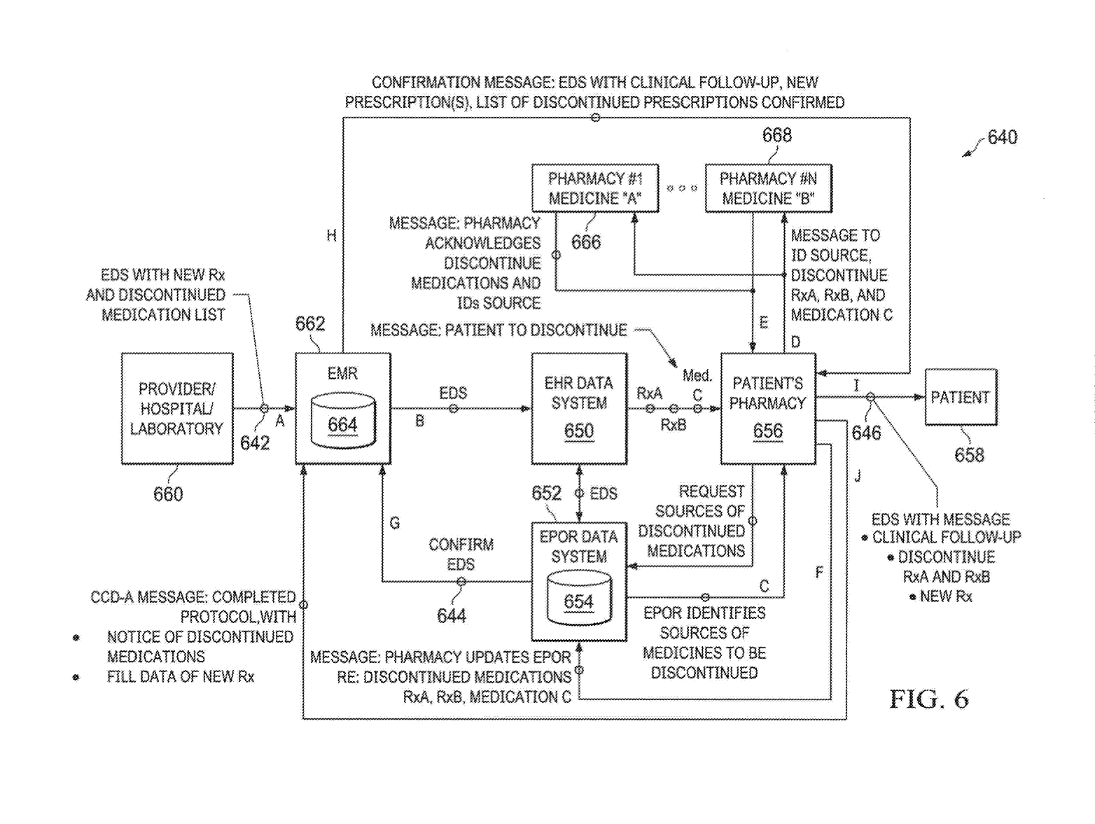

Another key feature of these embodiments is the use of the unique data file structure called a “clinical analytical message” or “CAM” that travels with the processing steps governed by software according to custom algorithms resident primarily in the clinical services platform. The CAM, which may be structured as depicted in FIG. 6 described herein, is used in both the submission review and the fulfillment review phases of the processing. These review phases are somewhat similar in concept but distinctly different in their structure and processes from the well-known “pre-edit” and “post-edit” processes used by pharmacies and payers in the industry to examine prescription claims for accuracy and consistency using established data and rules for processing claims.

As mentioned previously, in the processing of prescriptions for specialty drugs it is required to supply clinical and educational services information to the patient who is to receive specialty drugs. As will be described, the information needed to satisfy that requirement will be stored in the EPO database 206, and the instruction regarding that requirement will be contained in the CAM data file, or CAM 220 in FIG. 2. In this case, the CAM 220 may be configured as a “Pre-CAM 220A” for “pre-clinical analytic message,” that governs the submission review phase of the prescription processing. The source of the clinical and educational services information is typically based on data verified by physicians and medical schools or medical research institutions.

Also associated with processing specialty drug prescriptions is a “Post-CAM 220B,” a post-clinical analytic message, that governs the fulfillment review phase of the prescription processing. The post-CAM 220B preferably includes, among other data, information about when the specialty drug can be dispensed by the pharmacy to the patient, or in some cases administered to the patient by a licensed healthcare practitioner, along with the required clinical and educational services information.

### DETAILED DESCRIPTION OF THE INVENTION

In the description that follows of both processes for submission and fulfillment of a prescription, the prescription, a unit of information, is given the reference number 88, and a claim, also a unit of information, is given the reference number 90. Two specific types of claims will be described: the claims 90 include first claims 91 for delivery of the clinical and educational services via path 312 and second claims 92 for dispensing the drugs. The following description also includes additional embodiments of the invention: an electronic admission summary, a Medication Reconciliation Summary (see FIG. 4), and an Electronic Discharge Summary (EDS).

FIG. 1 illustrates a system network diagram depicting major functional components of one embodiment of the present invention. The healthcare data system 100 is structured around its operational connections with the Internet 102, which is accessed via a website portal 104 and provides links with participating entitles in the healthcare data system 100 via an electronic transaction hub 106. The electronic transaction hub 106 may be known in the industry as the “switch,” may be any of several entities, and will be referred to herein as the switch 106. Coupled to the Internet via first 112 and second 122 data processing links are first 110, second 120, and third 130 “cloud” systems. The cloud systems 110, 120, and 130 are shown as three separate entities because they each may contain the same data and processing and programming structures to provide essential redundancy and back up capability.

As is well known, a cloud may be defined as a system of computing resources—data storage. data processing, software, and communications components remote from user locations but accessible to the users on request. One principal utility of data processing facilities available in clouds is that users are freed of the burdens of installing and maintaining their own data processing resources. Instead, the necessary data processing components, including proprietary software stored in a cloud, can be accessed at will during the operation of the user's systems. Thus clouds may reduce the capital expense of maintaining one's own computing infrastructure to an operating expense. Another important advantage of clouds is the ease of providing redundant and secure resources for critical systems such as healthcare data systems that have substantial data backup and security requirements. Clouds may be public or private. Public-clouds may have multiple users who share the available resources. Private clouds are maintained by individual entities that limit access for their own purposes.

In FIG. 1, in one illustrative but not limiting configurations, the cloud 110 includes a first review processor 114, which may include proprietary software that fulfills an important function in the healthcare data system 100. Similarly, a second cloud 120, which may be a duplicate of cloud 110, provides system redundancy and may include a second review processor 124 and duplicate proprietary software. To illustrate that certain important segments of healthcare data can be stored in the first 110 and second 120 clouds, a third cloud 130 is depicted in FIG. 1, which may represent associated portions of the first 110 and second 120 clouds. In one example, the data storage functions of the third cloud 130 are shown accessible through a third data processing link 132 that connects them with the website portal 104. Also connected to the third data processing link 132 are the three databases: EPO Data 140, ePR Data 144, and the consolidated EPOR 150 database. Persons skilled in the art will recognize the other configurations are possible for implementing the system described herein.

The EPO data 140 may represent a database containing standardized files and data and services that may include but not be limited to 1) drug data; 2) laboratory data; 3) genomic data; 4) healthcare provider data; 5) data on collaborations among healthcare providers; 6) disease management information for both chronic and acute conditions; and data about 7) NHIN services and contracted sendees. NHIN is the abbreviation for the National Health Information Network, which provides for secure exchange of health information via the Internet using a common platform. The NHIN also establishes standards, policies and services for the use of the platform. The EPO data 140 may be accessed by public participants in the healthcare data system 100.

The ePR data 144, named an “electronic pharmacy record, may represent a central, intra-pharmacy database, or collectively a plurality of pharmacy databases linked together and associated with, for example, the pharmacy departments of chain retailers. The ePR database 144 may store patient and pharmaceutical data associated with prescription processing by participating pharmacists.

The EPOR 150 database may be a comprehensive, consolidated database containing at least one complete record of the patient's clinical and pharmaceutical data and may be structured and dedicated for use by participating members of the healthcare data system 100. It receives data and information updates with each healthcare transaction that takes place in and is processed by the system 100. Data and information are received from both the EPO data 140 and from the ePR data 144, which are not generally accessible to all providers Thus, whenever one of the participating providers authorized to log into the website portal 104 via the respective portals 162-174, the data accessible to them resides in the consolidated database called EPOR data 150.

Two groups of participants in the healthcare data system 100 are represented in FIG. 1. One group is coupled via provider portal links 160 to the website portal 104. This group of participants may include, but not necessarily limited to patients 162, physicians 164, pharmacies 166, hospitals and clinics 168, laboratories 170, and entities providing genomic and other types of medical research data 174. Providers of genomic data 172 and medical school research data 176 may be linked respectively to the laboratories 170 and the research entities 174.

FIG. 2 illustrates a functional block diagram of front end processing of a prescription for a so-called “specialty drug” according to one embodiment of the invention. The term “front end” processing refers to submission processing of the prescription for the specialty drug. So-called “specialty drugs,” are typically very expensive, require special handling, and prescribed for rare or debilitating diseases. The submission processing affects handling of the prescription—i.e., evaluation of the prescription in an automatic editing process—from entry of the prescription into the system by a healthcare provider (such as a physician) of a prescription for a patient to reception by a pharmacy selected to fulfill the prescription by dispensing the drug to the patient or a caregiver.

FIG. 2 depicts an example of the “front end” processing of a pharmaceutical prescription. The processing by submission review system 200 performs review of a prescription 88 submitted by a healthcare provider 204 such as a physician or nurse practitioner. The prescription 88 may be in real time via either of the paths 214, 222, or 224. Thus, when the prescription 88 is submitted by hitting the send key on the provider's terminal, the system 200 performs the submission review and returns, within a few seconds, either a confirmation that no edits to the prescription 88 are required or an instruction that the prescription 88 should be, or in some cases must be revised—edited—because of one or more factors that turned up during the submission review process that would find the initial prescription 88 unsuitable for its intended purpose or potentially capable of harm to the patient. In the following description, a prescription 88 is defined to be a unit of information identifying a drug prescribed by a provider, the “SIG,” a label that states instructions about when to administer the drug (including how often during each 24 hour period), and the quantity of the medication (typically specified in milligrams or “mg.”) This “script” information is used by the dispensing pharmacy to fill the prescription 88 and by the patient as the instructions in administering the drug.

At the heart of the submission review system 200 of FIG. 2 (and also the fulfillment review system 300 of FIG. 3) is a clinical services platform 202, which together with the Clinical Analytical Message 220 form a novel combination with the consolidated EPOR 150 database not heretofore available. These key components together solve the problems of integration of the disparate components and data formats in the healthcare industry as described herein. A patient safety portal, which provides for safe and secure patient access to that patient's electronic patient outcomes record EPOR 150 and provides the enabling authorization for clinical practitioners, is provided by the website portal 104 into the system 100 that facilitates much of the functionality of the present embodiment. The website portal 104 may include the interface into the interdisciplinary electronic patient outcomes record 150 (EPOR 150 database) from both the patient safety portal and interdisciplinary portals for each healthcare provider discipline shown coupled via the links 160 to the website portal 104 depicted in FIG. 1. Also shown coupled with the EPOR 150 database is the essential facilitating component, called the specialized “clinical services platform” 202 for the coordinating the operations of the EPOR 150 database configured to obtain and consolidate the data from the EPO data 140 and ePR 144 databases described above in the description of FIG. 1. The software program instructions stored in non-volatile memory in the clinical services platform 202, operating with the review processor 212, are configured to interface with and interact with the pharmaceutical delivery system, and among other operations, to provide electronic discharge summary reports—and access to them—as will be described.

The processing speed that enables the submission review system 200 to respond to the patient or provider in real time is provided by the combination of dedicated program instructions located within a virtual host or cloud and operated when called within the review processor 212 during submission review and upon direct access from the review processor 212 to the comprehensive EPOR 150 database. Having the wide range of patient, pharmaceutical, and insurance plan information assembled in one electronic location—the EPOR 150 database—and directly accessible by the review processor 212 enables the dedicated program instructions to perform the submission review processing with practically zero delay.

Operations performed in and by the review processor 212 include recording the prescription information entered by the provider 204 with the identity of the patient's preferred pharmacy, and analyzing the submitted electronic prescription to determine whether edits are needed to ensure that the prescription is appropriate and safe for the diagnosed disease and the patient. The review processor 212, upon completing its review, assembles the CAM 220 by populating its data fields with relevant data from the EPOR 150.

The analysis of the electronic prescription and the relevant data imported into the EPOR 150 from the EPO 140 and ePR 144 databases may be performed by an analytics engine situated in or closely coupled with the clinical services platform 202. The analytical engine gathers the healthcare data relevant to the submitted electronic prescription, organizes it using algorithmic operations to determine the efficacy and accuracy of the prescription for the patient and the particular diagnosed disease state or injury, and suggests or requires edits to the prescription if needed.

The prescription information may include new prescriptions and discontinued prescriptions. Using a file tag, the review processor 212 calls custom algorithms from the program instructions to analyze the populated data fields in the CAM 220 in light of the prescription 88 entered into the submission review system 200. The custom algorithms are also constructed to perform drug utilization reviews, analyzing the prescription for drug interaction with information in the EPOR 150 database such as (but not limited to) other drugs the patient may be taking, known food or other allergies, particular disease conditions of the patient, laboratory data from various tests (e.g., blood, urine, and other body fluids), patient disease and medical data, the patient's genomic profile, and provisions of the patient's health insurance that may be relevant to the particular prescription.

FIG. 3 illustrates a functional block diagram of back end processing of the prescription 88 for a specialty drug according to the embodiment of FIG. 1, and follows and is closely related to the embodiment of FIG. 2. Portions of FIG. 2 appear in FIG. 3; accordingly those structures of FIG. 2 bear the same reference numbers. For example, the switch 208 is shown with an input from the review processor 202, which in turn is coupled and has access to the contents of the databases shown in FIG. 2, namely: the EPO data 140, the ePR data 144, and the consolidated data in the EPOR 150 database. The term “back end processing” refers to the fulfillment processing of the prescription 88. The system is specifically configured for processing prescriptions 88 for specialty drugs. The fulfillment processing affects handling of the prescription 88 by the pharmacy beginning with receipt of an edited version of the prescription 88 submitted from the originating healthcare provider. The process of fulfillment includes providing clinical and educational services information to the patient, acknowledging that service, filling and dispensing the prescription drug to the patient, preparation of claims 90 for payment for those services, and processing of those payments to the manufacturer, distributor, and retail pharmacy by the payers on behalf of the patient.

The fulfillment processing begins when a prescription 88 (originated by a healthcare provider 204) sent from the review processor 202 with the clinical analytical message (CAM) 220 is received via path 212 and transmitted by the switch 208 over path 242 to the pharmacy 210 (See also FIG. 2). Note that for purposes of this description, the EMR 218 is assumed to be part of the path 214 in FIG. 3. The pharmacy 210 performs several functions. If the prescription 88 is for a specialty drug the pharmacy 210 reviews and prepares the prescription 88 for delivery of clinical and educational services at step 260 along paths 330 and 334 and dispensing or administering the prescription drug to the patient in step 302 through the step 250. The clinical and educational services information is contained in the CAM 220 associated with a specialty drug prescription. Upon delivery of these services via step 260, the CAM 220 confirms delivery of the services 260 along path 262. From there the clinical analytical message 220 travels via step 340 along path 264 via the switch 208 to update the EPOR 150 data thereby notifying the healthcare provider 204, and also to the manufacturer 360 of the specialty drug via the path 254, thereby notifying the manufacturer that the order for the specialty drug sent by the pharmacy 208 over the path 332 can be released. If the prescription is not for a specialty drug, no clinical or educational services are required and the pharmacy 210 dispenses the drug to the patient 302 at step 250.

Fulfillment processing includes processing of claims 90 for payments to the pharmacy 210 for services it renders. It also includes processing of payments to the manufacturer 360 of the specially drugs after it delivers the drugs to the inventory of a Group Purchasing Organization or structure 370 (aka “GPO 370”). The GPO 370 is established to serve a warehouse function by member pharmacies to maintain an inventory of specialty drugs and facilitate processing of payments and credits for its costs. The specialty drugs received from the manufacturer 360 along path 342 are delivered by the GPO 370 after the specialty drug manufacturer 360 receives the order from the pharmacy 210 along path 332. Confirmation of delivery 340 of the clinical and educational services to the patient via path 264 authorizes release of the specialty drug by the manufacture 360 via path 342 to the GPO 370, which delivers the specialty drug to the pharmacy along path 348.

Similarly, the path 342 represents the dual role of delivering, to order, the specialty drug from the manufacturer 360 to the GPO 370, and for sending the bill for the specialty drug to the GPO 370. Associated with and in response to receipt of the bill for the specialty drug by the GPO 370, followed by remittance of payment for the specialty drug along path 344 to the manufacturer 360, the manufacturer 360 may issue an electronic coupon (aka “eCoupon”) via path 346 that represents a discount to be credited to the patient's account at the pharmacy 210 for the cost of the specialty drug. The eCoupon 346 is processed by a Pharmacy Benefit Coalition or entity (aka “PBC”) 310 en route to the GPO 370 for delivery to the pharmacy 210. The PBC 310 acts as a coordinating entity for pharmaceutical benefits available from payers associated with the patient—usually an insurance company or employer by contract or benefit plan or Federal or State benefits such as Medicare and Medicaid.

The pharmacy benefit coalition or PBC 310 is an entity formed by the inventor of the present system as an efficient alternative to the industry's pharmacy benefit manager (or “PBM”). The PBC is devised to efficiently coordinate, adjudicate as necessary, and reconcile the payer benefits to the pharmacy on behalf of the patient by clearing e-prescriptions (“eRx”) for fulfillment and facilitating the transactions associated with fulfilling the e-prescription. The GPO 370 may also forward payment for the specialty drug to the manufacturer 360 via the path 344.

When the pharmacy 210 has delivered the clinical and educational services (for specialty drugs) and dispensed the specialty of non-specialty prescription drugs, and established acknowledgment of those services, the pharmacy 210 prepares and files claims 90 for payment with the PBC 310. The claims 90 include first claims 91 for delivery of the clinical and educational services via path 312 and second claims 92 for dispensing the drugs. The PBC then forwards the claims 91, 92 to a payer of record 320, which may adjudicate the claims 91, 92 according to its own rules and procedures before forwarding them to the PBC 310 to reconcile the required remittances and advise the PBC 310 as necessary before submitting the reimbursements along the path 324 to the pharmacy 210. The processing of these claims 91, 92 may also be characterized respectively as the first and second reimbursement transactions.

Turning now to FIG. 4, one example of the operation of the system of FIG. 1 is described in reference to FIG. 4 as follows. This example uses the capability of the system to enable a patient being discharged from a healthcare treatment episode, for example a hospital, a doctor's office, a clinic, a laboratory or imaging appointment, or even after receiving an immunization or certain clinical counseling at a pharmacy, to receive an up-to-date report on the status of his or her healthcare as it is affected by the visit to the facility that provided the healthcare services. In one important instance, a patient being discharged from a hospital stay is given a discharge summary that provides information that the patient needs during his or her recovery. This information may include information about prescription drugs, both new and continuing medications, discontinued prescriptions, laboratory tests that need to be completed, recommended therapies and care instructions, and identification and contact information for additional providers if applicable. This record provided to the patient is a “hospital discharge summary.” In the present embodiment this summary may also be called an electronic treatment discharge summary (“ETDS”) or an Electronic Discharge Summary (“EDS”).

The data for generating the EDS or ETDS resides in the EPOR 150 that is depicted in FIGS. 1, 2 and 3. The EPOR 150 is accessible through the website portal 104 by the patient. Thus, upon discharge, the patient is given the instruction, such as a wallet card, memory device or similar object, that includes an access code or password and his or her unique identifier to enable secure access to the patient record in the EPOR 150 database. Upon receipt of a request, the clinical services platform 202 may format the EDS or ETDS for download to the patient at an electronic address provided by the patient. The electronic address may be an email address, a cell phone or other mobile number, etc. The file may typically be formatted as a PDF, or as a data file that may be transferred to a portable storage medium. The portable storage medium may be one of a limited number of choices available to the patient.

Referring to FIG. 4, an alternate embodiment of the invention depicted in FIGS. 1, 2 and 3 is configured for providing reports generated from the consolidated EPOR 150 database. It is one of a variety of examples of healthcare information that may be provided to a patient or a provider merely by accessing the website portal 104 of FIG. 1. Many of the structures of FIG. 4 are the same as shown in FIGS. 1 and 2, but drawn to illustrate the process of producing and delivering an electronic discharge summary 410 (“EDS” 410) or an electronic admission summary 420 (“EAS 420”). The block in FIG. 4 for the EDS 410 may be adapted functionally to furnish the EAS 420 or any other report such as a medication reconciliation report and the like.

Expressed more generally, the electronic discharge summary 410 may be called an electronic treatment summary because it may be configured to report important information to the patient on discharge or release from hospitals as well as physician's offices, laboratory or imaging facilities, and the like. In the case illustrated a hospital electronic discharge summary may contain information about (a) new prescriptions (new scripts); (b) discontinued scripts; (c) inferred ICD-10 data; and (d) discharge “CMR,” which is information about comprehensive medication review. Instead of the traditional hard paper copy of this report given to the patient upon discharge from a hospital, the patient may be given an access key or password to enable request for the electronic discharge summary through the website portal 104 of the National Healthcare Coalition 400 as depicted in FIG. 1. The electronic discharge summary (“EDS”) 410 may be generated from data stored in the consolidated EPOR 150 database according to a formatting application in the clinical services platform 202 of the Healthcare Data Network.

Continuing with FIG. 4, upon discharge or release of a patient 162 from a hospital 168 or physician 164, or other treatment, imaging, laboratory or prescribing facility (not shown), the hospital, physician, or other facility communicates information about the treatment or services rendered into the system for processing by the clinical services platform 202. By communicating through the National Healthcare Coalition 400 healthcare data in the EPOR 150 may be updated in real time. The EPOR 150 may also receive pharmaceutical data updates regarding prescriptions, new or discontinued, from one or more electronic Pharmacy Records, ePR #1 through ePR #N. The clinical services platform 202, utilizing the review processor 212 (see FIGS. 2 and 3) embodied therein, processes the data updates into the EPOR 150 and forwards the pharmaceutical updates to a pharmacy 166 indicated in the EPOR 150. The clinical services platform 202 also formats the electronic discharge summary 410 specific to the patient 162 at the time of the patient's need and request. The request may be enabled by a data access code and password issued to the patient 162 at the time of discharge or release.

In another important example of the utility of the EPOR 150 database, the system is capable of providing a comprehensive medication reconciliation summary or report to providers or patients upon admission for evaluation or treatment by the provider. An up-to-date patient medication history, including current prescriptions, discontinued drugs, and over-the-counter medicines is essential to both the admission process and to safe prescribing of new medications by a provider or any other change to a patient's medications. The present invention provides both real time updating of all patient records and the ability to prepare the Medication Reconciliation reports or other summaries or reports on demand substantially at the time of admission for evaluation or treatment.

Referring again to FIG. 4 and the box 410, which maybe adapted and redefined to furnish a Medication Reconciliation & Patient Record 430, the process will be described as follows. It is a similar process to the case illustrated for a hospital electronic admission summary that may contain information about (a) new prescriptions (new scripts); (b) discontinued scripts; and (c) other information useful for the review of medications for safety and accuracy of the prescriptions. Instead of the traditional hard paper copy of this report that may be prepared to the provider upon admission of a patient to a hospital or physician, for example, the provider may be given an access key or password to enable request for the electronic admission summary through the website portal 104 of the National Healthcare Coalition 400 as depicted: in FIG. 1. The electronic admission summary (“EAS”) 420 may be generated from data stored in the consolidated EPOR database according to a formatting application in the clinical services platform 202 of the Healthcare Data Network.

Continuing with FIG. 4, upon admission of a patient 162 to a hospital 168 or physician 164, for evaluation, treatment, imaging, laboratory or prescribing facility (not shown), the hospital, physician, or other provider communicates information about the reason(s) for the admission into the system for processing by the clinical services platform 202. By communicating through the National Healthcare Coalition 400 the healthcare data in the EPOR 150 is updated in real time. The EPOR 150 also receives pharmaceutical data updates regarding prescriptions, new or discontinued, from one or more electronic Pharmacy Records, ePR #1 through ePR #N. The clinical services platform 202, utilizing the review processor 212 (see FIGS. 2 and 3) embodied therein, processes the data updates into the EPOR 150 and forwards any pharmaceutical updates to the electronic admission summary (or medication reconciliation report) or to a pharmacy 166 indicated in the EPOR 150. The clinical services platform 202 also formats the electronic admission summary 420 specific to the provider 164, 168 substantially at the time of the patient's admission. The request is enabled by a data access code and password issued to the provider 164, 168 at the time of admission.

Persons of skill in the art will recognize that the system described herein forms a new combination of new structures with existing structures operative in the healthcare industry. The new system helps providers comply with Federal mandates to provide a safe, efficient, and secure patient outcomes record that enables objective measurement of the outcome of episodes of care received by patients. The new structures include a system of an associated electronic patient outcomes record (EPOR), a new clinical services platform, and a new clinical analytical message data file. The system is organized around the systems, processes, and databases for prescribing and verifying (submission), and fulfilling drug prescriptions, providing clinical data and counseling, reconciling the associated payment transactions, extracting pharmaceutical and clinical information to compile electronic discharge and admission reports, and providing real time updates to the stored data at every step of the processes. Structural features of the system are linked through individual portals with healthcare providers and which also permit access by individual patients through a dedicated, secure portal into the system and the electronic patient outcomes record (EPOR). These structural features include a variety of new software mechanisms within the clinical services platform, a National Health Information Network (NHIN), and a pharmaceutical delivery system that are configured for communication such that portability and interoperability of the all healthcare-related data in the system is ensured.

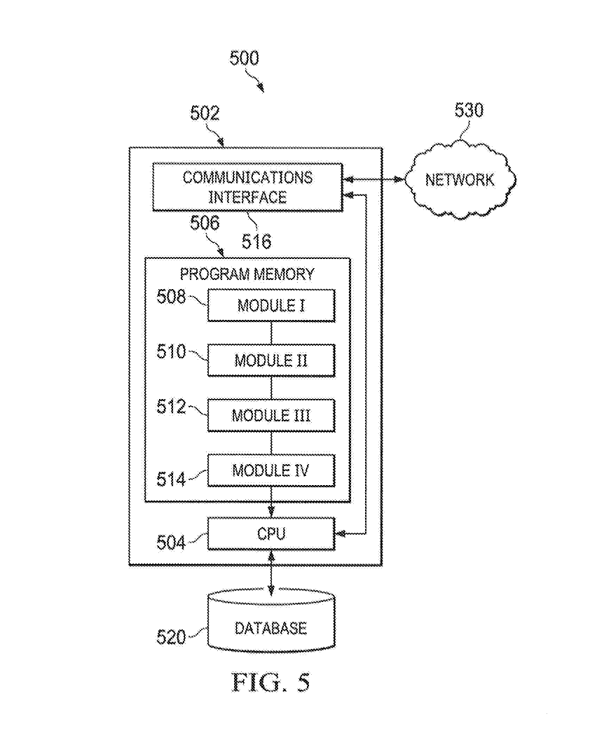

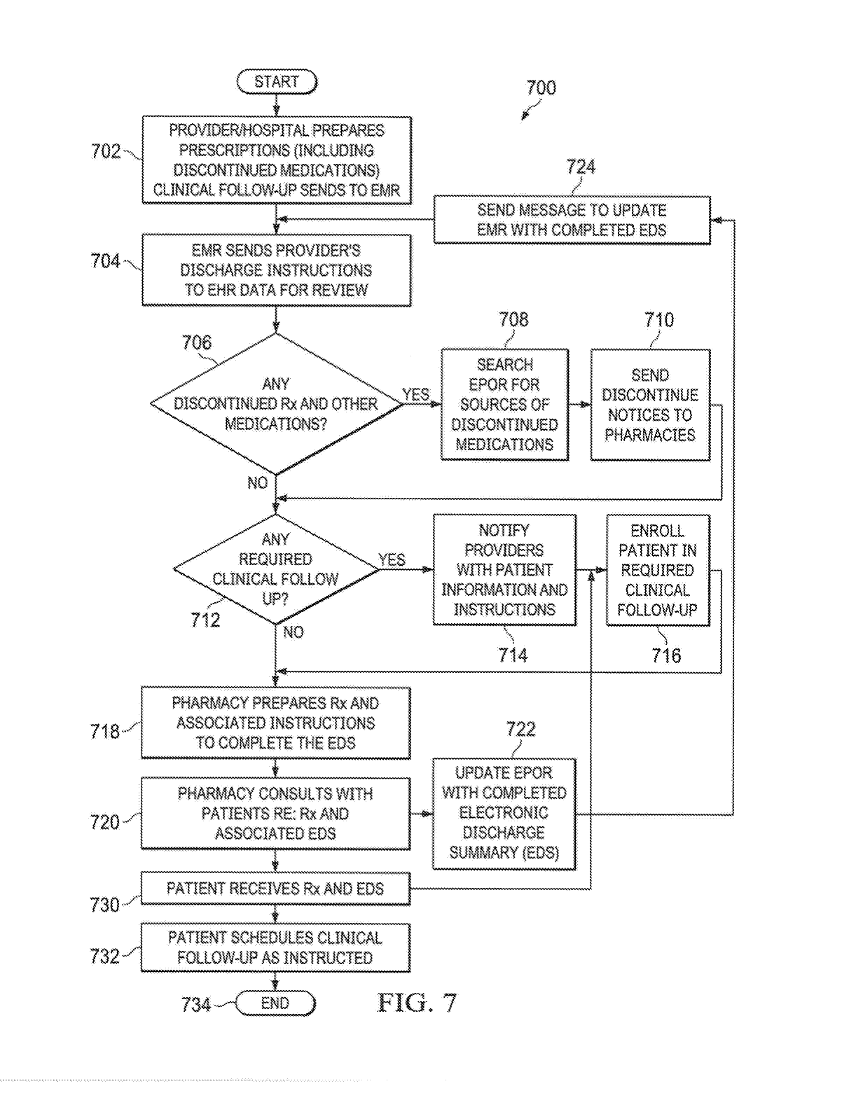

FIGS. 5, 6, and 7 illustrate one alternate embodiment of a system and process for providing an electronic discharge summary (EDS) to an identified patient upon discharge front a healthcare treatment facility. The EDS may be prepared or delivered by a healthcare provider such as a physician, a nurse practitioner, or an assistant under the supervision of a physician. In general, a discharge summary is a document that contains information about a diagnosis of an identified patient's condition, a new prescription, a list of discontinued medications, and instructions for additional laboratory tests and clinical follow-up such as appointments with other physicians or healthcare facilities. It is understood that the term medications refers to both prescription medicines and non-prescription medications. Included in FIG. 6 and 7 are capital letters A through J, which generally correspond to the sequence of operations performed in the system.

The present invention in this alternate embodiment provides a system and process for preparing and delivering an electronic version of—and a replacement for—the information typically presented as a hard page document to the patient upon discharge from a hospital, a physician's office or other type of healthcare facility. The invention includes processes for ensuring the accuracy of the EDS, that medications and prescriptions to be deactivated and discontinued are properly communicated and recorded in the identified patient's prescription records, and that the EDS itself is properly and timely communicated to the identified patient.

FIG. 5 illustrates a block diagram of a data processor according to an embodiment of the present invention. The data processing system 500 includes a data processor 502 that may at least comprise a CPU 504, a program memory 506, and a communication interface 516. The program memory 506 may include a plurality of program modules or application program interfaces (APIs) for performing various functions when called by the operating system (not shown as it is a well-known component of a CPU or central processing unit). A module I may be configured as a graphical user interface for displaying information needed by a user. A module II may be configured for control of a first data processor configured as in FIG. 5 or, alternately, as a module III for control of a second data processor as will be described. A module IV may be configured for processes to control confirmation and communication of the EDS within the system and from the first pharmacy to the patient identified on the EDS. The graphical user interface may be configured to display the information content of the EDS at various stages of the processes being carried out, and to facilitate edits or messages associated with the reviews and processes involving the EDS. The data processing system 500 may be coupled for exchanging data with a database 520; and the data processor 502 is enabled to communication with an external network 530 vis the communication interface 516.

FIG. 6 illustrates a system block diagram of the alternate embodiment of the present invention. The diagram depicts the structure of the system 640 that includes brief legends to clarify functional steps being carried out by the system components. The patient's pharmacy 656, the first data processor (EHR Data System 650, and the second data processor (EPOR Data System 654) are the principal components of the inventive embodiment forming the claimed system. The system interacts and communicates with other, external entities as shown in FIG. 6. For example, the patient's pharmacy 656—also called the first pharmacy 656 herein—will be enabled to review prescription records of the identified patient with a prescription history record in other pharmacies 666, 668 associated with the first pharmacy 656 and having prescription records of the identified patient 658.

Continuing with FIG. 6, the provider/hospital/laboratory 660 prepares a discharge summary in electronic form—the electronic discharge summary (EDS) 642 that it communicates via an electronic medical record (EMR) intermediary 662 to the EHR Data System 650 (i.e., the first data processor) for processing. The EDS 642 generally contains the patient's identity, and information about a diagnosed condition, a new prescription, a list of medications (previously prescribed or of non-prescription medications) to be deactivated and discontinued, and instructions for clinical follow-up. The clinical follow-up instructions may include without limitation appointments with other physicians or specialists, admission to a hospital, rehabilitation facility, specialized patient care, or to a laboratory for further tests, etc.

Upon receiving the EDS 642 from the EMR 662, which may include access to a database 664, the first data processor 650 reviews the received EDS 642 for the content thereof, particularly the list of medications to be discontinued, designated in FIG. 6 as Rx_{A}, Rx_{B}, and Med_{C}. The first data processor—the EHR data system 650—may be configured by first programmed instructions to receive the EDS 642 issued by the healthcare provider 660 via the EMR intermediary 662 and process instructions in the EDS 642 to discontinue the medications to be deactivated by comparisons with first information regarding previous prescriptions dispensed to the identified patient 658 received from a network of other pharmacies 666, 668 including the first pharmacy 656 that have access to prescription records of the identified patient 658 and with second information from a consolidated database 654 in an electronic patient outcome record (EPOR) data system 652. The “second information” obtained from the consolidated database 654 may include prescription records for the identified patient 658 as well as comprehensive, standardized pharmaceutical, laboratory, genomic, disease state and other clinical data relevant to the EDS undergoing processing in the present system.

The consolidated database 654 forms part of the second data processing system 652. In operation, the second data processor 652 coupled to the consolidated database 654 and to the first data processor 650, and configured by second programmed instructions to receive the first and second information about the identified patient's 658 prescription history from the network of pharmacies 666, 668 and from the consolidated database 654, to confirm that medications to be deactivated are discontinued, and to provide the EDS 642 to the EMR 662 for transmission to the first pharmacy 656 for dispensing the new prescription and the EDS 642 to the identified patient 658. For example, communication between the first pharmacy 656 and the consolidated database 654 enables the system to identify the sources of the medications to be deactivated and discontinued, followed by a message containing the updates to the identified patient's 658 pharmacy records. Similarly, the first pharmacy also may prepare a CCD-A (consolidated clinical document architecture) message for transmission back to the EMR 662 that the required protocol involving the EDS 642 has been completed including notice of the discontinued medications and the fill data of the new prescriptions to be dispensed. This information is forward from the EMR 662 to the first pharmacy 656 for processing of the new prescription, dispensing the new prescription, and delivering the EDS 642 to the patient 658.

FIG. 7 illustrates a flow chart of an example of one process 700 that can be carried out in the embodiment of FIGS. 5 and 6. The process begins with step 702 wherein the healthcare provider such as a hospital, after preparing and compiling new prescriptions and clinical follow-up information in a discharge summary document to be delivered to a patient being discharged, sends the document in electronic form—an electronic discharge summary 642—to the EMR 662. In step 704 the EMR 662 sends the EDS to the EHR data system, i.e., the first processor 650 for review. The review may include several steps. For example, in step 706, the review determines whether there is a listing of any medications—prescriptions or non-prescription medications—to be deactivated or discontinued. If a list is found (YES), the flow advances to step 708 to search the EPOR data system 652 for the sources of previous medications, thence to step 710 to send discontinue notices to pharmacies in the network of pharmacies 666, 668, and then to step 712 to determine whether any clinical, follow-up is required. If YES, providers with patient information are notified accordingly along with required instructions, and the process proceeds to step 716 to enroll the patient in the required clinical follow-up procedure or visit to a healthcare entity. The flow then returns to the main path at step 718. If the determination in step 706, or in step 712, was negative, the process advances to step 718.

In step 718, the first pharmacy 656 (also: patient's pharmacy 656) prepares the new prescription contained in the EDS 642 along with the required clinical follow-up and other instructions to be delivered to the patient 658, followed by dispensing the new prescription and the EDS 642 in step 720. This step may include consultation by a pharmacist. The patient receives and acknowledges receipt of these items in step 730 and either notes appointments already scheduled on his or her behalf or schedules those appointments for further treatment, tests, or consultations himself or herself in step 732, and the process flow ends at step 734. If the patient 658 has further questions the process may return to step 716 and repeat the process at the first pharmacy 656. Following the step 720 for the consultation regarding the EDS and the new prescription, the flow advances to step 722 to update the consolidated database 654 in the EPOR data system 652 with the completed delivery of the EDS 642 and dispensing of the new prescription. A message to update the EMR 662 may follow in step 724. Thereupon the process may end or proceed to process another EDS 642 submitted by a provider.

In the alternate embodiment described above as shown in FIGS. 5, 6 and 7, persons of skill in the art will recognize that a new architecture is disclosed that solves the problem of providing an electronic discharge summary that includes timely notice and verification that previous medications prescribed for and taken by a patient that are required to be discontinued by a healthcare provider are properly and timely delivered to the patient and that all applicable records of prescriptions and other medications are correctly updated in all relevant databases and patient records. The individual elements and steps of the claims of this invention for the respective system and process disclosed herein are not well-understood, routine, or conventional. Instead, the claims are directed to the unconventional inventive concept described in this specification.

### TECHNOLOGICAL SUMMARY

The present invention provides a first new system architecture for processing healthcare data that is depicted in FIGS. 1, 2, and 3. The invention comprises a combination of data processing mechanisms arranged and configured for efficiently processing both prescription submissions and fulfillment, as well as healthcare data updates and safe, secure patient outcome data accessibility in real time to all authorized healthcare providers and patients.

The technological improvements in the first embodiment of the present invention include configuring the following three innovations: (1) an enhanced database organization system; (2) an enhanced (automated) prescription submission and fulfillment processing system provided by a new clinical services platform; and (3) a clinical analytical message for facilitating data movement, notification, and processing in both systems (1) and (2). A further improvement in a second embodiment is the availability of a discrete reports of patient records such as the electronic discharge summary EDS 642 (See, e.g., FIG. 6) or an electronic admission summary or EAS 420 (See, e.g., FIG. 4), and, more generally an electronic Medication Reconciliation summary 430 that may be provided to a provider substantially at the time of admission (i.e., in real time) into a hospital, or a provider such as a physician's office, laboratory, or imaging facility. Moreover, the system may also provide a discrete electronic discharge summary EDS 642 or EDS 410—more generally an electronic treatment summary—that may be provided to a patient upon request at the time of discharge or release (i.e., in real time) from a hospital, or in other examples, from a clinic, physician's office, laboratory, or imaging facility.

Returning now to the system of FIGS. 1, 2 and 3 that includes the ability to provide the EDS to the patient as depicted in FIG. 4, the healthcare data processing system draws its data from two principal databases, the ePR (pharmaceutical data) and EPO data (standardized healthcare data) databases, both of which contain volumes of data formatted in disparate configurations from multiple sources.

Part of the job of populating the third principle database—the consolidated EPOR database containing a complete patient healthcare outcome record—includes translating the disparate formats of the source data into a common format (such as XML, for example) for the EPOR database. Moreover, the EPOR database may, in some embodiments include a data processor configured with program instructions for processing the data to be stored therein or accessed from the EPOR database. The clinical services platform contains the mechanisms for (A) translating these data, (B) installing them in an organized way in the EPOR database; (C) maintaining the record in the EPOR updated with every prescription processing and other healthcare provider transaction; and (D) enabling real time, interoperable access available to all authorized providers and patients of data updated with every healthcare transaction. An important ingredient of the processing system that facilitates the movement of data in the functions of the clinical services platform is the clinical analytical message (“CAM”), which in some embodiments maybe configured as a data file.

Accordingly, a novel patient-centric, portable, interoperable, interdisciplinary, electronic patient outcome record and system, accessible in real time by authorized healthcare providers and their patients through a website portal into the system, is provided by the present invention. The system comprises the novel combination of a single healthcare database, a clinical services platform,: and a clinical analytics message data file. Each of these three innovations in the technology of healthcare data processing are specifically configured for their respective data processing functions. The advantage of this approach is that the system could not otherwise be configured to perform its functions in real time because the compromises in security, and speed and resulting bottlenecks associated with modified traditional structures are eliminated. The efficiencies resulting from the dedicated architecture and speed—and the absence of bottlenecks—engineered into the system are essential to provide a system capable of responding to the extraordinary growth of healthcare data needs now and in the foreseeable future. It is this combination, and the unique characteristics of each of these elements working together that provides the secure, real time access and processing of prescription drug submission and fulfillment transactions associated with treatment of a patient by a healthcare provider. Moreover, the system as thus constructed provides enhanced patient safety and security of healthcare information while providing up-to-date patient outcome data in compliance with federal regulations.

The consolidated healthcare database is populated with pharmaceutical and standardized healthcare data at every prescription transaction, including translation of the native data format of the source data into a common, readily accessible data format. The clinical services platform, through its interactions with the active components of the system, using the suite of mechanisms both structural and algorithmic directs the processing for both constructing and maintaining the single healthcare database and for processing the submission and fulfillment of prescriptions submitted by the patient's healthcare providers. The data transactions and processes carried out by the clinical services platform with its review processor, operating in conjunction with the single healthcare database, are enabled and facilitated in real time by a unique clinical analytical message data file that conveys both data and process commands within the system and with external entities such as a transaction hub (the “switch”), claim processing entities, payers, and drug manufacturers.

Persons skilled in the art will readily understand that these advantages and objectives of this system that provides real time, interoperable updates and access to a patient's secure healthcare data would not be possible without the particular combination of computer hardware and other structural components and mechanisms assembled in this inventive system and described herein. It will be further understood that a variety of programming tools, known to persons skilled in the art, are available for implementing the control of the features and operations described in the foregoing material. Moreover, the particular choice of programming tool(s) may be governed by the specific objectives and constraints placed on the implementation plan selected for realizing the concepts set forth herein and in the appended claims.

While the invention has been shown in only one of its forms, it is not thus limited but is susceptible to various changes and modifications without departing from the spirit thereof. For example, each of the new structures described herein, such as the EPOR database, the clinical services platform, the review processor, and the CAM, as a message and as a data file, may be modified to suit particular local variations or requirements while retaining their basic configurations or structural relationships with each other or while performing the same or similar functions described herein.

In another example, a potential use for the CAM or the CAM in the form of a data file may be during the submission review process to determine whether the submitted prescription is for a specialty drug that requires delivery of specific clinical and educational information to the patient or is not for a specialty drug, which ordinarily does not require such specific information be delivered to the patient. A feature of the CAM data file in that instance may be to identify drugs with certain associated risks that suggest the patient be counseled by the pharmacy to identify patient-specific factors or vulnerabilities to those risks.

In the embodiments described herein, for example as shown in FIGS. 1, 2, and 3; in FIG. 4; and in FIGS. 5, 6 and 7, persons of skill in the art will recognize that a new architecture is disclosed in these embodiments that solves the problems of (1) automatically and in real time evaluating a prescription for efficacy for the patient based upon a comprehensive review of all available pharmaceutical and healthcare data in a thorough, pre-edit review process carried out during the submission of a new prescription before it is received by a pharmacy for fulfillment; and of (2) providing an electronic discharge summary that includes timely notice and verification that previous medications prescribed for and taken by a patient that are required to be discontinued by a healthcare provider are properly and timely delivered to the patient find that all applicable records of prescriptions and other medications are correctly updated in all relevant databases and patient records. The individual elements and steps of the claims of this invention for the respective system and process disclosed herein are not well-understood, routine, or conventional. Instead, the claims are directed to the unconventional inventive concepts of the embodiments described in this specification.

What is claimed is:

1. A system for providing an electronic discharge summary (EDS) to an identified patient upon discharge from a healthcare treatment facility, comprising:
a first pharmacy for receiving an EDS that includes a new electronic prescription for a pharmaceutical issued by a healthcare provider in the healthcare facility via an electronic medical record (EMR) intermediary to a first data processor, wherein the EDS, after a review by the first data processor, is forwarded to the first pharmacy to compare medications to be deactivated by the EDS with prescription records of the identified patient with a prescription history record in other pharmacies associated with the first pharmacy and having prescription records of the identified patient;
a first data processor configured by first programmed instructions to receive the EDS issued by the healthcare provider via the EMR intermediary and process instructions in the EDS to discontinue the medications to be deactivated by comparisons with first information regarding previous prescriptions dispensed to the identified patient received from a network of other pharmacies including the first pharmacy that have access to prescription records of the identified patient and with second information from a consolidated database in an electronic patient outcome record (EPOR) data system; and
a second data processor coupled to the consolidated database and to the first data processor, and configured by second programmed instructions to receive the first and second information about the identified patient's prescription history from the network of pharmacies and from the consolidated database, to confirm that medications to be deactivated are discontinued, and to provide the EDS to the EMR for transmission to the first pharmacy for dispensing the new prescription and the EDS to the identified patient.

2. The system of claim 1, wherein the first pharmacy is the pharmacy identified by the identified patient, i.e., the patient's pharmacy.

3. The system of claim 1, wherein the medications to be deactivated includes previous prescription and non-prescription medications.

4. The system of claim 1, wherein the EPOR data system includes the second data processor.

5. The system of claim 1, wherein the EDS includes at least one new prescription, a list of medications to be discontinued, and clinical follow-up instructions.

6. The system of claim 1, wherein the healthcare provider includes a physician, a nurse practitioner, or other authorized healthcare professional in the healthcare facility.

7. The system of claim 1, wherein the first and second processors respectively comprise:
a central processing unit having an operating system;
a non-volatile program memory containing a plurality of program instruction modules; and
a communication interface for connecting and operating with an external network.

8. The system of claim 7, wherein the program instruction modules include:
a first module containing a graphical user interface;
a second module containing the first programmed instructions;
a third module containing the second programmed instructions; and
a fourth module containing dispensing and consultation procedures.

9. The system of claim 7, wherein the external network comprises:
a first link via an EMR to the healthcare facility;
a second link from the first pharmacy to the network of other pharmacies; and
a third link from the first pharmacy to the EMR.

10. A process for providing an electronic discharge summary (EDS) to an identified patient upon discharge from a healthcare treatment facility, comprising the steps of:
receiving the EDS and at least one new electronic prescription included therein for a diagnosed condition of the identified patient as issued by a healthcare provider in the healthcare facility in the name of the identified patient and transmitted via an electronic medical record (EMR) intermediary to a first data processor configured by first programmed instructions;
reviewing in the first data processor according to the first programmed instructions the EDS to identify the at least one new electronic prescription and medications listed in the EDS to be deactivated and to confirm the presence of clinical follow-up instructions associated with the diagnosed condition stated in the EDS;
parsing the contents of the EDS in the EPOR into its components and forwarding each component to a first pharmacy for comparing the medications to be deactivated with first information regarding previous prescriptions dispensed to the identified patient received from a network of other pharmacies including the first pharmacy that have access to prescription records of the identified patient, and with second information from a consolidated database in an electronic patient outcome record (EPOR) data system.
sending notices to the network of other pharmacies that the previous prescriptions and medications included in the list of medications to be deactivated are to be discontinued;
notifying healthcare providers to schedule appointments for providing the clinical follow-up according to the clinical follow-up instructions in the EDS;
preparing the new prescription and the accompanying EDS for dispensing to the patient; and
comparing the EDS with the first and second information in a second data processor configured according to second programmed instructions to confirm that medications to be deactivated are discontinued, and to provide the EDS to the EMR for transmission to the first pharmacy for dispensing the new electronic prescription and the EDS to the identified patient.

11. The process of claim 10, wherein the step of reserving comprises the step of:
acknowledging receipt of a message containing the EDS to the EMR.

12. The process of claim 10, wherein the step of reviewing comprises the step of
confirming presence of required components of the EDS including patient identity, condition diagnosed, new prescription and non-proscription medications, medications to be deactivated and discontinued, and clinical follow-up instructions.

13. The process of claim 10, wherein the step of parsing comprises the steps of:
accessing a module from the programmed instructions in the first data processor to compare a medication to be deactivated with the first information regarding previous prescriptions; and
comparing the medication to be deactivated with the second information from the consolidated database.

14. The process of claim 10, wherein the step of notifying comprises the step of:
enrolling the identified patient in the required clinical follow-up according to the clinical follow-up instructions.

15. The process of claim 10, wherein the step of preparing the new prescription comprises the steps of:
filling the new prescription for dispensing it to the identified patient; and
delivering the EDS and the new prescription and associated consultation as needed about the new prescription, the deactivated medications, and the clinical follow-up instructions contained in the EDS.

16. The process of claim 10, wherein the step of comparing comprises the steps of:
confirming to the EPOR data system the comparison of the EDS with the first and second information; and
updating the EPOR data system with a message stating the new prescription has been dispensed, the discontinued medications deactivated, and the clinical follow-up instructions delivered and scheduled.
